(19) United States
(12) Patent Application Publication
(10) Pub. No.: US 2021/0389315 A1
(43) Pub. Date: Dec. 16, 2021

(54) POINT OF CARE DEVICE FOR EARLY AND RAPID DISEASE DIAGNOSIS

(71) Applicant: Case Western Reserve University, Cleveland, OH (US)

(72) Inventors: Susann Brady-Kalnay, Cleveland, OH (US); Robert Deissler, Fairview Park, OH (US); Robert Brown, Solon, OH (US)

(21) Appl. No.: 17/347,892

(22) Filed: Jun. 15, 2021

Related U.S. Application Data
(60) Provisional application No. 63/039,317, filed on Jun. 15, 2020.

(51) Int. Cl. G01N 33/543; G01N 33/569

(52) U.S. Cl. CPC G01N 33/54333; G01N 2333/165; G01N 33/54373; G01N 33/56983

(57) ABSTRACT

Early stage, rapid, low-cost, and accurate detection of disease components in a biological fluid is critically important. A point of care device can use functionalized magnetic beads to facilitate this detection. The device can include a sample holder with a collection region. A magnet can be used to draw the functionalized nanoparticles bound to the disease component into the collection region, where the disease component is captured. A light source can shine a light beam through the collection region; and a detector can detect the light beam after traversing the collection region to determine whether the disease component is present in the sample.

### CROSS-REFERENCE TO RELATED APPLICATIONS

This application claims the benefit of U.S. Provisional Application No. 63/039,317, filed Jun. 15, 2020, entitled “A POINT OF CARE DEVICE FOR ANTIBODY OR VIRAL DETECTION AND CAPTURE: CAPTIV”. This provisional application is hereby incorporated by reference in its entirety for all purposes.

### TECHNICAL FIELD

This disclosure relates generally to disease diagnosis and, more specifically, to a point of care device that uses functionalized magnetic particles coated with recognition components to facilitate detection and capture of the disease components in a biological fluid.

### BACKGROUND

COVID-19 is the disease caused by the pathogenic coronavirus SARS-CoV-2. A variety of new diagnostic approaches have been developed (using genomic tools (e.g., RT-PCT assays) and molecular probes) that can identify patients suffering from COVID-19 by detecting the SARS-CoV-2 virus or a body's antibody response to the SARS-CoV-2 virus. However, detecting the SARS-CoV-2 virus or a body's antibody response to the SARS-CoV-2 virus using these new diagnostic approaches may require days or weeks beyond the first exposure. When a COVID-19 infection can be detected in its earliest stages, fewer people will be exposed to the SARS-CoV-2 virus, thereby slowing the spread of COVID-19.

### SUMMARY

Early stage, rapid, low-cost, and accurate detection and capture of disease components is critically important in disease diagnosis and its eventual treatment. For example, early detection of viruses and/or anti-viral antibodies is vital with pathogens, like SARS-CoV-2. Furthermore, as new mutant strains emerge it is necessary to both detect and capture these mutant strains. In addition, capture of the antibodies produced in response to a COVID-19 infection could be developed into novel therapeutics for the treatment of SARS-CoV-2. The present disclosure relates to a point of care device that uses functionalized magnetic particles coated with recognition components to facilitate detection and capture of the disease components in a biological fluid.

In accordance with an aspect of this disclosure, a system is provided that can detect and capture certain disease components in a biological fluid. At least a portion of the system can include a diagnostic device that can be used as a point of care device for the detection and capture of disease components. The system includes a sample holder comprising: a binding region configured to hold a sample and a plurality of functionalized magnetic particles, each of the plurality of functionalized magnetic particles being linked to a recognition component configured to bind to a disease component within the sample to form clusters; and a collection region configured to collect and capture the disease component therein. At least one magnet can be configured to provide a magnetic field gradient that draws the clusters from the binding region into the collection region. A light source can be on one side of the collection region configured to shine a light beam through the collection region; and a detector can be on an opposite side of the collection region from the light source configured to detect the light beam after the light beam has traversed the collection region to determine whether the disease component is present in the sample based on the detected light beam.

In accordance with another aspect of this disclosure, a method is provided for detecting and capturing certain disease components in a biological fluid. The method includes functionalizing magnetic particles to link to a certain recognition component, wherein the certain recognition component is configured to bind to the disease component; linking a plurality of the functionalized magnetic particles to one or more of the certain recognition component; adding the functionalized magnetic particles linked to the one or more of the certain recognition component to a sample holder that holds a sample, wherein the certain recognition component binds any disease component in the sample to form clusters; drawing the clusters into a collection region of the sample holder with a magnetic field gradient; and capturing the clusters in the collection region of the sample holder. The disease component can be detected in the collection region.

In accordance with another aspect of this disclosure, another method is provided for detecting and capturing certain disease components in a biological fluid. The method includes adding a sample to a sample holder, wherein the sample comprises functionalized magnetic particles linked to the one or more recognition components, wherein the one or more recognition components are configured to bind to disease components in the sample; providing a magnetic field gradient configured to draw any clusters of the recognition component and magnetic particles bound to disease component into a collection region of the sample holder; and shining light through the collection region of the sample holder, wherein a change in intensity of the light through the collection region is indicative of a presence of the disease component in the sample.

### BRIEF DESCRIPTION OF THE DRAWINGS

The features, objects, and advantages of the invention will become more apparent from the detailed description set forth below when taken in conjunction with the drawings, wherein:

FIG. 1 is a block diagram of an example system that can be used to detect and capture certain disease components in a biological fluid;

FIG. 2 is a block diagram of another example system that can be used to detect and capture certain disease components in a biological fluid;

FIG. 3 is a block diagram of another example system that can be used to detect and capture certain disease components in a biological fluid;

FIG. 4 is an illustration of functionalized magnetic particles being coated with recognition components;

FIGS. 5 and 6 are illustrations of what happens when functionalized magnetic particles coated with recognition components are placed in a sample without a disease component (FIG. 5) and with a disease component (FIG. 6);

FIGS. 7 and 8 are illustrations of what happens when functionalized magnetic particles coated with recognition component are placed in a sample without a disease component (FIG. 7) and with a disease component (FIG. 8) and exposed to a magnetic field;

FIGS. 9 and 10 are process flow diagram of example methods for detecting and capturing certain disease components in a biological fluid.

### DETAILED DESCRIPTION OF THE INVENTION

This disclosure relates generally to disease diagnosis based on early stage, rapid, low-cost, and accurate detection and capture of disease components in biological fluid. This disclosure also relates to the capture of disease components for further analysis. In some examples, the disease components can be isolated from a sample and captured for further study. In other examples, the disease components can be either detected then captured or captured then detected.

The disease components can be present at a low level, undetectable by traditional means. The disease component can be an antibody, a virus, a bacterium, a crystal, an exosome, a cell from a cancerous tissue, etc. In one example, the disease component can be a coronavirus, a type of virus that causes a disease, such as COVID-19, SARS, or MERS. The biological fluid (also referred to as a “biofluid”) can be any type of fluid or tissue (which can be placed within a fluid that may or may not originate from the body) originating from an organism (e.g., bacteria, fungi, plant, human or animal) that is known to house the disease component. Different disease components can be housed in different biofluids. Biofluids can be excreted (such as sputum, nasal excretions, urine or sweat), secreted (such as breast milk), obtained with a needle (such as synovial fluid, blood, or cerebrospinal fluid), or develop as a result of a pathological process (such as blister fluid or cyst fluid). Cell culture media can also be a type of biofluid. As used herein, a “sample” can be a portion of biofluid being tested to see if a certain disease component can be detected therein.

More specifically, this disclosure relates a point of care device (referred to as “CAPTIV”) that can utilize magnetic particles, magnets, a light source, and a detector to detect and capture disease components. More specifically, CAPTIV uses functionalized magnetic particles (which may also be referred to as magnetic beads) coated with recognition components to facilitate detection and capture of the disease components in a biological fluid. As one example, a functionalized magnetic particle can be a magnetic particle that has had a linker molecule attached to its surface in order to modify the physical and/or chemical properties so that one or more recognition components can bind to and/or coat the magnetic particle. The linker molecule can be any molecule that is functionally attached (e.g., covalently linked) to a magnetic particle that creates an adhesion point for a recognition component. The recognition component can be a viral protein, an envelope associated cellular protein, a proteinase, a coat protein, an envelope protein, a spike protein, an antibody, an antibody fragment, a peptide, a nucleic acid, or the like. As an example, the recognition component can be an Fc chimera protein such as ACE-2-Fc, TMPRSS2-Fc, GRP-78-Fc, DC-SIGN-Fc, or DC-SIGNR-Fc. As another example, the recognition component can be a native and/or a recombinant protein, like one of M, E, S, N, HE, 3, 6, 7, 8, 9, 10, NSP and ORF proteins. In fact, the protein can be a viral associated protein derived from infected cells. As a further example, the recognition component can be a nucleic acid, including at least a portion of RNA or DNA. As another example, the magnetic particles can be functionalized with the recognition component without requiring a linker molecule. As a further example, recognition components can be previously bound to disease components and then the recognition component can be attached to the functionalized magnetic particles. The examples can be referred to as “magnetic particles coated with recognition components” herein. The functionalized magnetic particles coated with recognition components can facilitate an amplification effect through clustering of attached magnetic particles. As used herein, a “cluster” can include a plurality of magnetic particles, a plurality of recognition components, and one or more disease components bound together. A cluster can be formed when at least one recognition component, attached to a functionalized magnetic particle, binds to a disease component. Due to their larger size than a single disease component alone, clusters can allow for disease components to be captured and detected at smaller numbers than traditional detection schemes using a magnetic gradient.

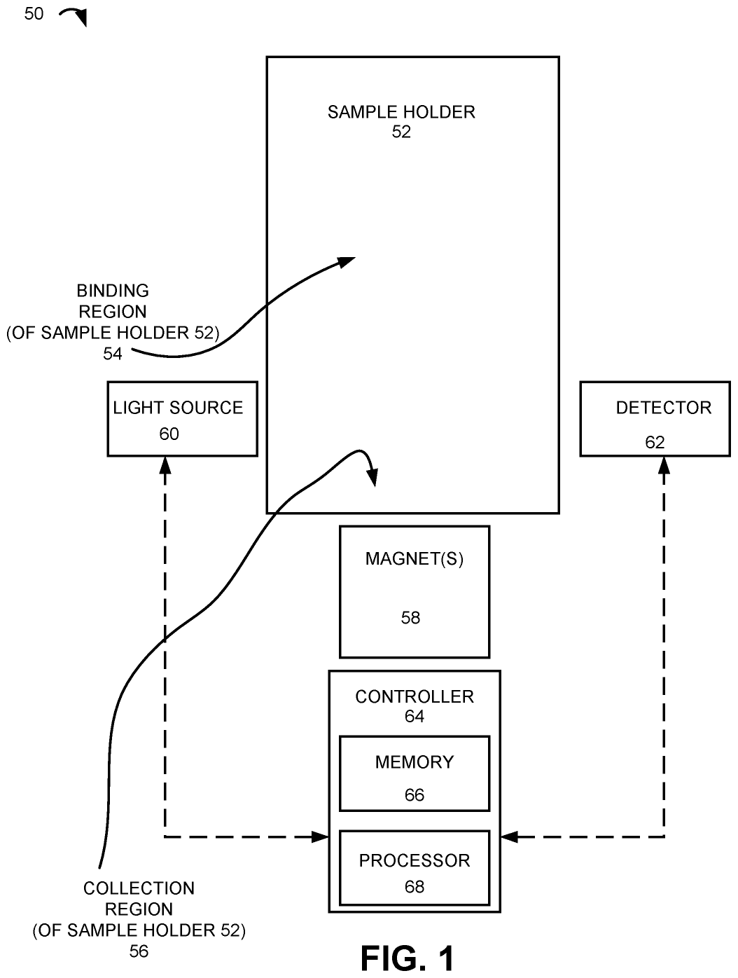

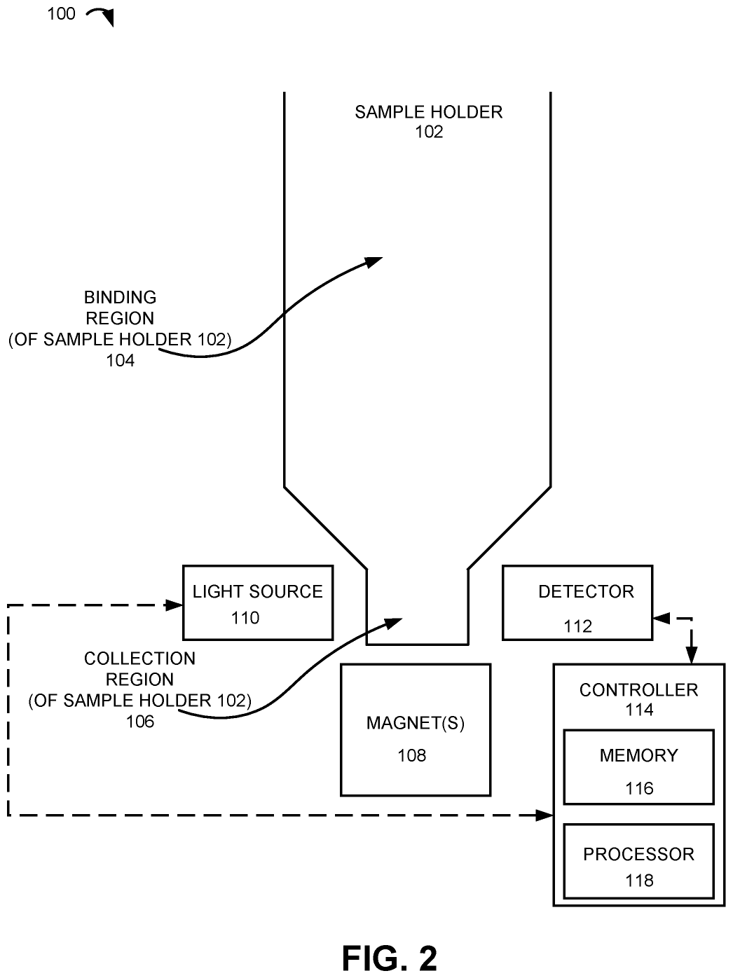

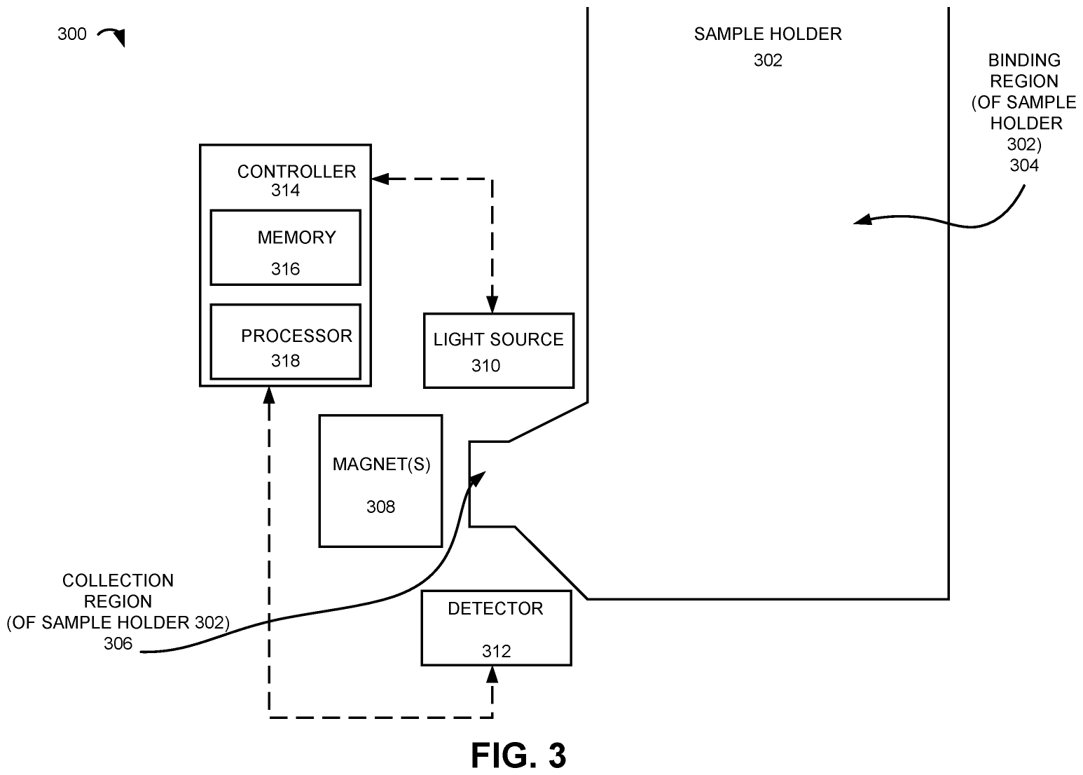

An example configuration of an example system 50 that can be used to detect and capture certain disease components in a biological fluid is shown in FIG. 1. An example of a configuration of an example system 100 that can be used to detect and capture certain disease components in a biological fluid is shown in FIG. 2. Another example of another alternate configuration of an example system 300 that can be used to detect and capture certain disease components in a biological fluid is shown in FIG. 3. At least a portion of the systems 50, 100 and/or 300 can be part of CAPTIV. CAPTIV can be a point of care device that can be used in a doctor's office, at a patient's home, in an emergency room, at a pharmacy, etc. However. It should be understood that CAPTIV can also be used in a laboratory setting.

The systems 50, 100 and 300 can each be used (1) to detect and capture disease particles in a biofluid sample and/or (2) capture the disease particles for further analysis. The detection by the systems 50, 100 and 300 is very sensitive due to the formation of clusters by functionalized magnetic particles coated with recognition components and disease components when the recognition components bind with one or more disease components. Advantageously, the disease particles can be captured while coupled to the recognition component, or after release from the recognition component, so that the disease component can be further studied in a “patient-derived” approach that allows analysis of the nuances or specificity of the disease component (e.g., as part of a cluster) in a biofluid sample of a particular patient.

The systems 50, 100 and 300 each include a sample holder 52, 102, 302 that can be configured to hold a sample (e.g., a portion of a biofluid). The biofluid can be any type of fluid or tissue (which can be placed within a fluid that may or may not originate from the body) originating from a living organism (e.g., human or animal) that is known to house the disease component. Different disease components can be housed in different biofluids. The sample holder 52, 102, 302 can be made of one or more transparent or translucent materials, such as one or more plastic, glass, or a combination of one or more plastic and glass. As an example, at least a portion of the sample holder 52, 102, 302 can be a cuvette. It should be noted that although the sample holder 102, 302 is illustrated as having rectangular/cubed shapes, this is for ease of illustration; the sample holder 52, 102, 302 can have one or more rounded edges or be other shapes (e.g., elliptical, triangular, polygonal, etc.).

The sample holder 52, 102, 302 can include a binding region 54, 104, 304 and a collection region 56, 106, 306. The binding region 54, 104, 304 can be configured to hold the sample (which may include a disease component), while the collection region 56,106, 306 can be configured to collect and capture a disease component therein. As shown in FIG. 1, the collection region 56 can be along the edges of the binding region 54 of the sample holder 52 and/or against one or more of the edges of the sample holder 52 (it should be noted that although collection region 56 is only shown against the bottom of the sample holder 52, the collection region 56 can be against any one or more edge of the sample holder). As shown in FIG. 2, the collection region 106 can be located below the binding region 104. However, the collection region need not be located below the binding region and may be located in a different area relative to the binding region. As shown in FIG. 3, for example, the collection region 306 is located off to a side of a part of the binding region 304. In some instances, the binding region 54, 104, 304 can have a larger interior volume than the collection region 56, 106, 306. In FIGS. 2 and 3, the binding region 104, 304 and the collection region 106, 306 can be contiguous parts of a single sample holder 102, 302 that shrinks down or tapers from the binding region 104, 304 to the collection region 106, 306. In another instance the binding region 104, 304 and the collection region 106, 306 of FIGS. 2 and 3 can each be in one or more separate sample holders 102, 302 that are in fluid communication with the binding region 104, 304 having a larger interior volume than the collection region 106, 306. For example, the binding region 104, 304 and the collection region 106, 306 can employ microfluidics in the design of the sample holder(s) 102, 302.

Functionalized magnetic particles coated with recognition component can be added to the sample held within sample holder 52, 102, 302 to facilitate the capture and/or detection of disease components. A functionalized magnetic particle can be a magnetic particle that binds directly or has had a linker molecule attached to its surface in order to modify the physical and/or chemical properties so that one or more recognition components can bind to and/or coat the magnetic particle. The linker molecule can be any molecule that is functionally attached (e.g., covalently linked) to a magnetic particle that creates an adhesion point for a recognition component. The recognition component can be a viral protein, an envelope associated cellular protein, a proteinase, a coat protein, an envelope protein, a spike protein, an antibody, an antibody fragment, a peptide, a nucleic acid, or the like.

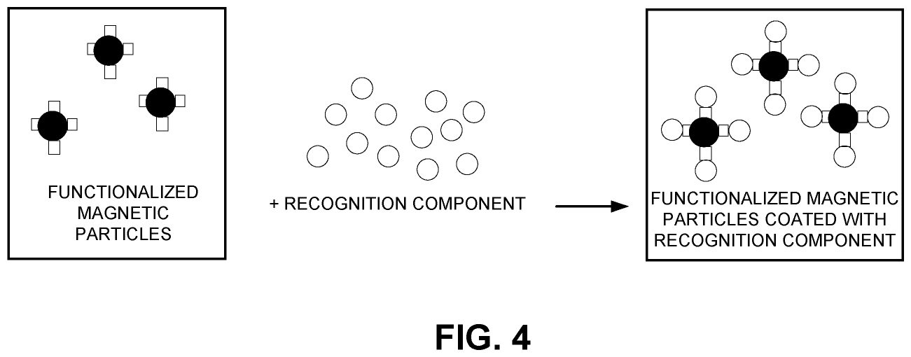

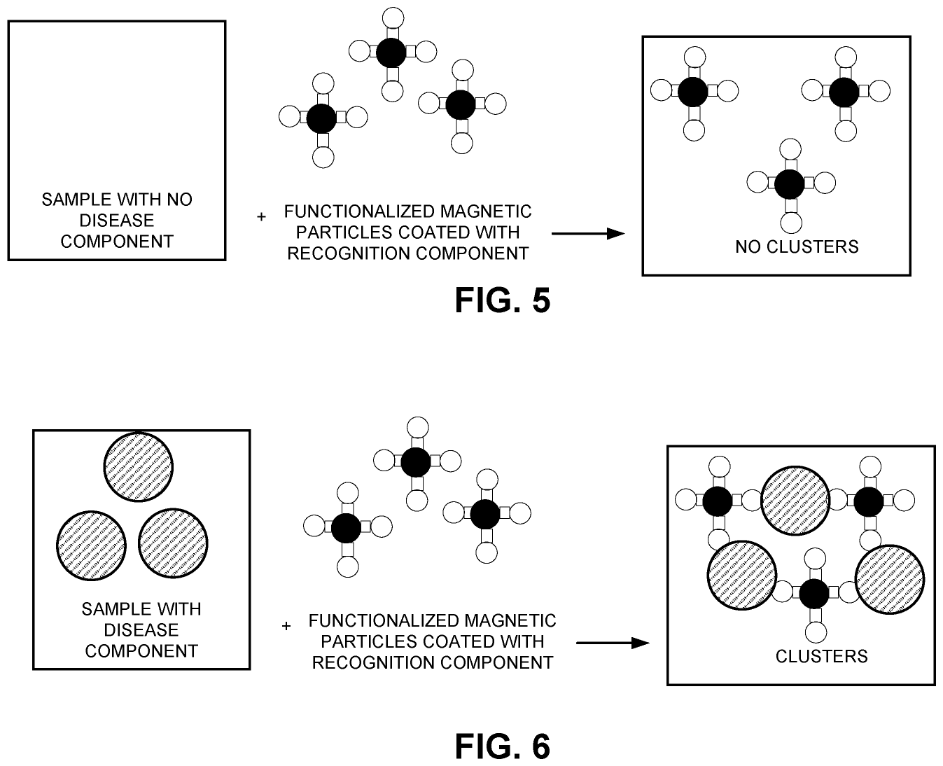

Specifically, the functionalized magnetic particles coated with recognition component can be added to the binding region 54, 104, 304 of the sample holder 52, 102, 302. As shown in FIG. 4, before the functionalized magnetic particles are added to the sample holder 52, 102, 302, the functionalized magnetic particles are coated with recognition component. In some instances, the recognition component can be selected based on the target disease component. In other instances, the recognition component can be selected generally to detect a variety of different disease components. The disease component can be an antibody, a virus, a bacterium, a fungus, a crystal, an exosome, a cell from a cancerous tissue, etc. Within the binding region 54, 104, 304, the functionalized magnetic particles coated with recognition component can attach to any disease component that the recognition component targets. If the sample contains none of the targeted disease component, then the functionalized magnetic particles coated with recognition components will not attach to anything. As shown in FIG. 5, when no disease component is within the sample, no clusters are formed. However, as shown in FIG. 6, when disease components are present within the sample, clusters are formed.

As noted, the functionalized magnetic particles coated with recognition component can attach to the disease component to form one or more clusters. Clusters can include a plurality of magnetic particles, a plurality of recognition components, and one or more disease components bound together. The clusters can be self-assembled. For example, one or more recognition components can attach to a disease component that is a virus through receptor mediated viral binding and/or viral protein attachment mechanisms, so that the disease component has one or more functionalized magnetic particles attached to it.

As shown in FIG. 5, when no disease component is within the sample, no clusters are formed. However, as shown in FIG. 6, when disease components are present within the sample, clusters are formed. Clusters can be as small as a single disease component with at least one functionalized magnetic particle attached via a recognition component. Preferably the smallest clusters can include a single disease component with at least two functionalized magnetic particles each attached to the disease component via a recognition component. Clusters can also be of a larger size, including a plurality of disease components connected with each other via bindings to a plurality of recognition components coating a plurality of functionalized magnetic particles, such as shown in FIG. 6. However, it should be noted that a cluster only needs many functionalized magnetic particles and does not need to include more than one disease component. Clusters allow for disease components to be captured and detected at smaller numbers than traditional detection schemes (i.e., the clusters amplify the detection capability for smaller numbers of disease components). Indeed, the magnetic force on the clusters due to the magnetic field gradient is much greater than the force on an individual magnetic particle.

Referring back to FIGS. 1, 2, and 3, one or more magnets 58, 108, 308 can be positioned to establish a magnetic field gradient that can draw the clusters into the collection region 56, 106, 306. It should be noted that the magnet(s) 58 in FIG. 1 can be positioned on any side of the sample holder 52. For example, the one or more magnets 58, 108, 308 can include a single magnet that is polarized to be north/south, with the north side closer to the collection region 56, 106, 306 and the south side away from the collection region 56, 106, 306. The one or more magnets can be positioned on an opposite side of the collection region 56, 106, 306 from where the collection region and the binding region 54, 104, 305 are connected (in fluid communication). It will be understood that different configurations of the one or more magnets 58, 108, 308 are possible as long as they establish the magnetic field gradient that can draw the clusters into the collection region 56, 106, 306. The magnets 58, 108, 308 can be moveable or able to be shielded, in some instances, so that after the clusters are pulled into the collection region 56, 106, 306, the disease component, at least a portion of the clusters can be collected. In some instances, the collection region 56, 106, 306 can be a microfluidic channel that can trap the clusters therein.

The one or more magnets 58, 108, 308 can include at least one simple, inexpensive lab magnet. However, the one or more magnets 58, 108, 308 can also include a permanent magnet. Generally, permanent magnets can produce a high magnetic field with a low mass. Additionally, a permanent magnet is generally stable against demagnetizing influences. For example, this stability may be due to the internal structure of the magnet. The permanent magnet can be made from a material that is magnetized and creates its own persistent magnetic field. The permanent magnet can be made of a hard ferromagnetic material, such as alcino or ferrite. However, the permanent magnet can also be made of a rare earth material, such as samarium, neodymium, or respective alloys.

As another example, the one or more magnets 58, 108, 308 can include an electromagnet. An electromagnet can be made from a coil of a wire that acts as a magnet when an electric current passes through it, but stops being a magnet when the current stops. The coil can be wrapped around a core of a soft ferromagnetic material, such as steel, which greatly enhances the magnetic field produced by the coil. For example, the magnetic field can be between about 0.01 T and about 100 T. As another example, the magnetic field can be between about 0.1 T and 10 T. As a further example, the magnetic field can be between 0.1 T and 2 T.

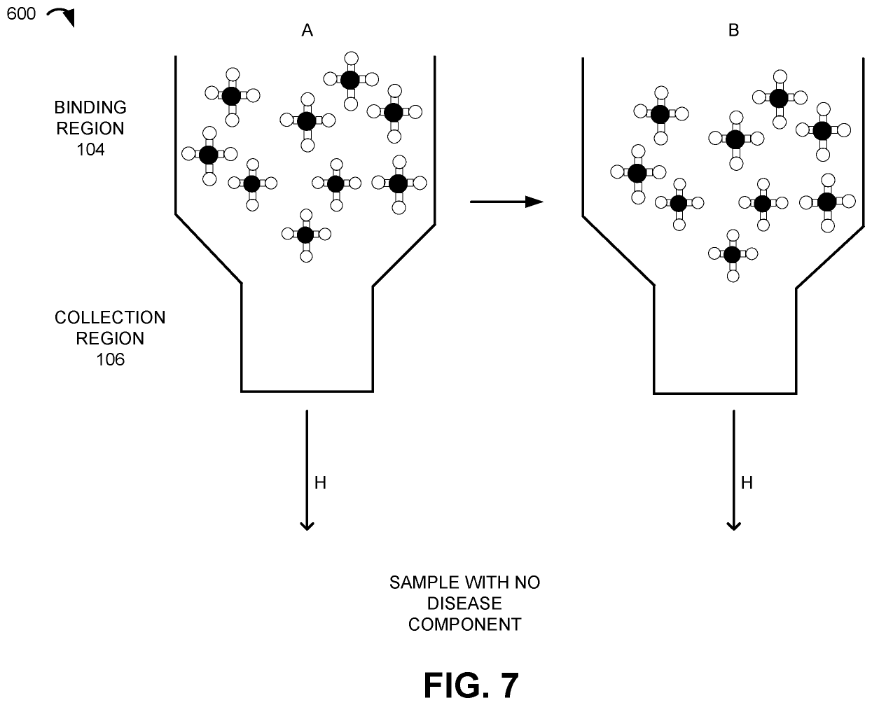

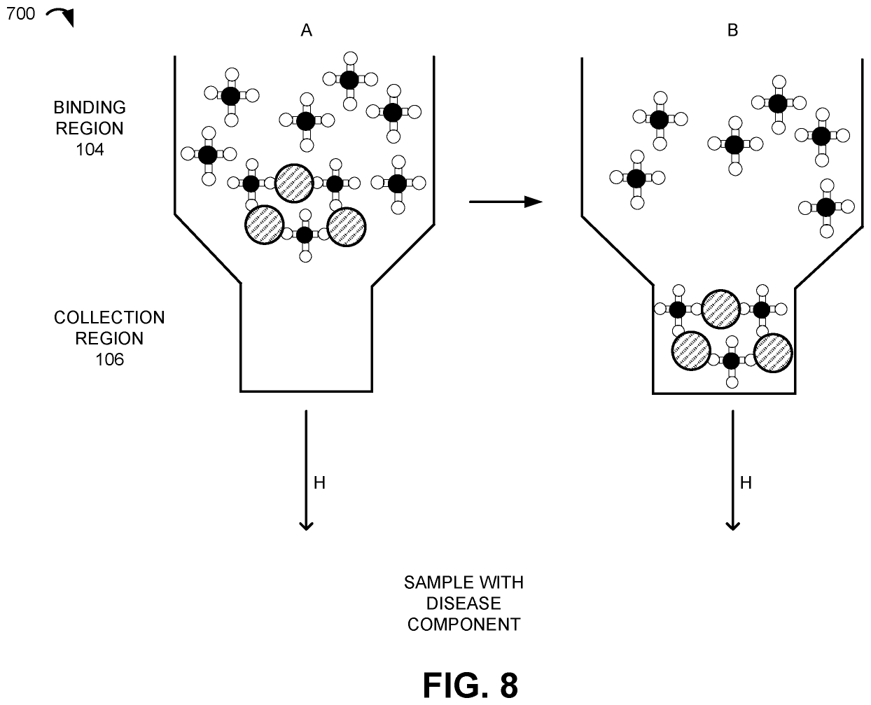

In operation, unbound functionalized magnetic particles coated with recognition component are not drawn into the collection region 56, 106, 306, while the disease component becomes bound to the functionalized magnetic particles through the recognition component and forms clusters that are drawn into the collection region 56, 106, 306. This is shown in FIGS. 7 and 8. When no disease components are within the sample (FIG. 7), the functionalized magnetic particles coated with recognition component do not form clusters (A) in the binding region 54, 104, 304 and are not substantially drawn into the collection region 56, 106, 306 (B). As an example shown in FIGS. 7 and 8, no unclustered functionalized magnetic particles coated with recognition component are drawn into the collection region.

The magnetic particles (functionalized or functionalized and coated with recognition component) are affected by the magnetic field gradient, but not to the same extent as clusters (e.g., single magnetic particles that are not part of a cluster also move under the magnetic field gradient, but to a lesser degree than those of the cluster). Larger magnetic particles are more affected by the magnetic field gradient than smaller magnetic particles, for example, at one magnetic field gradient strength 10 nm individual magnetic particles would not be pulled into the collection region 56, 106, 306 . To keep the majority of non-clustered magnetic particles out of the collection region 56, 106, 306 distinct field gradient strengths may be utilized for different size particles. The larger clusters are pulled towards the collection region 56, 106, 306 at a greater speed or acceleration than the individual particles. In another example, different size magnetic particles may be used. When disease components do exist in the sample (FIG. 8), one or more clusters are formed, and the clusters are drawn into the collection region 56, 106, 306. Any functionalized magnetic particles coated with recognition component that have not attached to a disease component are not drawn into the collection region 56, 106, 306. The magnetic field exerts a greater magnetic force on clusters that contain more than one magnetic particle than on single functionalized magnetic particles. For example, the more magnetic particles in a cluster, the greater the force of the magnetic field urging the clusters to the collection region 56, 106, 306.

The disease components can be detected within the collection region 56, 106, 306 or as the disease components are pulled into the collection region 56, 106, 306. In some instances (as illustrated), a light source 60, 110, 310 resides on one side of the collection region 56, 106, 306, while a detector 62, 112, 312 resides on an opposite side of the collection region 56, 106, 306. However, the light source 60, 110, 310 and the detector 62, 112, 312 need not be on either side of the collection region 66, 106, 306 and instead can be on either side of a different portion of the sample holder 52, 102, 302 where the clusters can be detected specifically (e.g., just before the collection region 106, 306 where the recognition portion narrows or tapers).

The light source 60,110, 310 can be configured to shine a light beam through the collection region 56, 106, 306 towards the detector 62, 112, 323. The light source 60, 110, 310 can also be configured to shine a light beam through any portion of the sample holder 52, 102, 302. For example, the light source 60, 110, 310 can provide coherent light and/or non-coherent light. The light source 60, 110, 310 can be a laser, an LED, a light bulb, or the like. The detector 62, 112, 312 can be configured to detect the light beam after the light beam has traversed the collection region to determine whether the disease component is present in the sample based on the detected light beam. The detector 62, 112, 312 can also be configured to detect fluorescence when the light beam passes through magnetic particles or disease components that have been fluorescently tagged. For example, the detector 62, 112, 312 can be a photodetector.

It will be noted that the light source 60, 110, 310 and/or the detector 62, 112, 312 (and in some instances the one or more magnets 58, 108, 308) can be wired to a controller 64, 114, 314 or other computing device, which can be used to operate the light source 60, 110, 310 and/or the detector 62, 112, 312 (and in some instances the one or more magnets 58, 108, 308) in at least a partially automated fashion. For example, the controller 64, 114, 314 or other computing device can regulate delivery of light, recording of data (e.g., sampling the detector 62, 112, 312), data analysis, configuration of the one or more magnets 68, 108, 308, or the like. The controller 64, 114, 314 can include a memory 66, 116, 316 storing instructions (that may be pre-programmed) and a processor 68, 118, 318 configured to access the memory 66, 116, 316 and execute the instructions. The controller 64, 114, 314 or other computing device can, in some instances, be connected to a display to visualize the collection region 56, 106, 306, the calculation, or the like.

As an example, the light source 60, 110, 310 can emit the light at an intensity. The detector 62, 112, 312 can detect the light at another intensity (which may be higher or lower). For example, the intensity change may be due to blocked light, fluorescence, or the like. The controller 64, 114, 314 can determine a difference (or an absolute value of the difference) between the emitted light and the detected light. If the difference is greater than a predefined threshold (e.g., which can be established as any number greater than 0, but may account for any error due to the detection mechanism and one or more materials of the sample holder 52, 102, 302, or one or more additional factors), a presence of the disease component can be detected. This detection may be confirmed by the capture of disease components.

In some instances, the sample within the sample holder 52, 102, 302 may be combined with a fluorescent tracker (e.g., a lipophilic dye) in order to tag any disease components therein. Fluorescent molecules of the fluorescent tracker may bind to the disease components in the sample (this may occur by adding the fluorescence tracker before the sample is placed in the sample holder 52, 102, 302 or after the sample is placed in the sample holder 52, 102, 302 at any point before the detection). Additionally or alternatively, a different fluorescent tracker (e.g., a different color, fluoresces at a different wavelength of light, etc.) can be added to the magnetic particles. The fluorescent tracker can fluoresce under the light beam and the fluorescing disease components can be detected by the detector 62, 112, 312 using traditional fluorescence detection methods. In other instances, the clusters can block light emitted by the light source 60, 110, 310 from reaching the detector 62, 112, 312.

The systems 50, 100, 300 have a greater sensitivity of detection than other previous schemes in an inexpensive form. Advantageously, the systems 50, 100, 300 also permit capture of at least the disease component for follow up studies. The collection region 56, 106, 306 can be used to facilitate the capture of disease components for further testing and analysis. As noted, the one or more magnets 58, 108, 308 can move or be shielded to facilitate the capture. In one example, the clusters can be captured and removed from the collection region 56, 106, 306. In another example, the clusters captured in the collection region 56, 106, 306 can be washed to remove the functionalized magnetic particles coated in recognition components (e.g., the wash can break the connection between the recognition components and the disease components). The isolated disease components can then be collected, for example with a micro-pipette, for further testing and/or follow-up studies, allowing for a patient-specific approach to isolate and analyze the molecular properties of an individual patient's disease component.

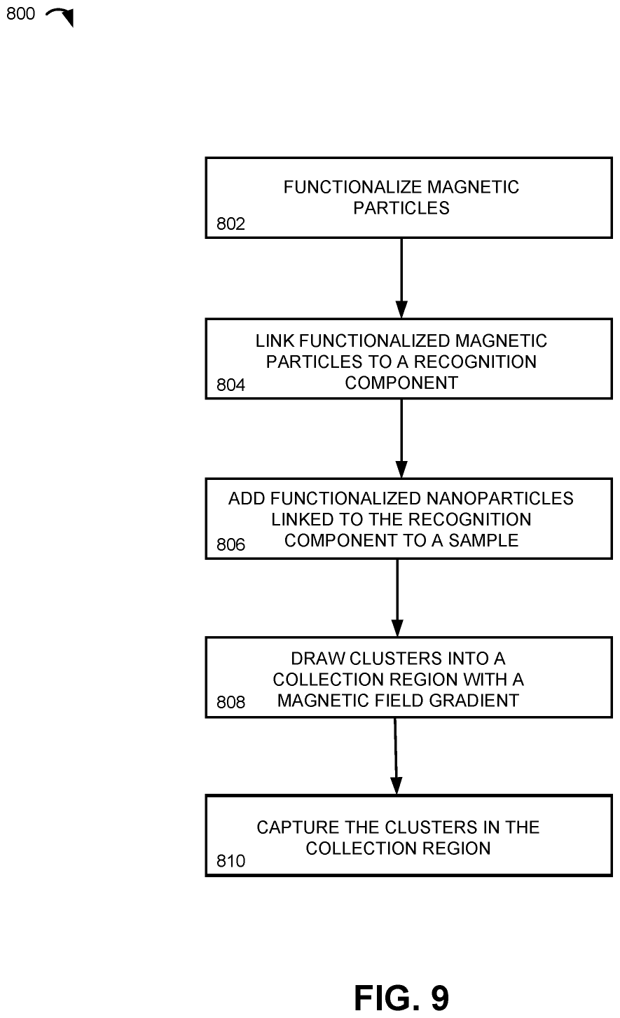

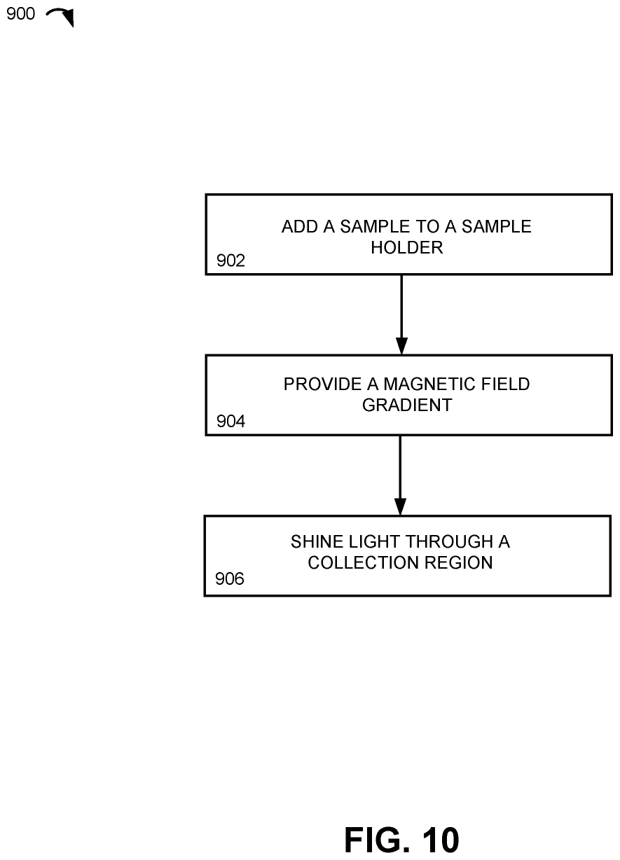

In view of the foregoing structural and functional features, example methods will be better appreciated with reference to FIGS. 9 and 10. While, for purposes of simplicity of explanation, the methods of FIGS. 9 and 10 are shown and described as executing serially, it is to be understood and appreciated that the present invention is not limited by the illustrated order, as some actions could, in other examples, occur in different orders from that shown and described herein or could occur concurrently. It will be appreciated that some or all acts of methods 800 and 900 can be implemented as machine-readable instructions on a non-transitory computer readable medium.

FIG. 9 illustrates an example method 800 for detecting and capturing certain disease components in a biological fluid. At step 802, magnetic particles can be functionalized to link to a certain recognition component. The certain recognition component is configured to bind to a disease component (e.g., a specific disease component, a class of similar disease components, etc.). At step 804, a plurality of the functionalized magnetic particles can be linked to one or more of the certain recognition components (e.g. shown in FIG. 4). At step 806, the functionalized magnetic particles linked to the one or more of the certain recognition components can be added to a sample. For example, the sample can be held in a sample holder (e.g., sample holder 52, 102, 302). Each certain recognition component binds a disease component in the sample to form clusters if the disease component is in a sample (the difference between having no disease component versus disease components in the formation of clusters is shown in FIGS. 5 and 6).

At step 808, the clusters can be drawn into a collection region (e.g., collection region 56, 106, 306 of sample holder 52, 102. 302). For example, the collection region can be exposed to a magnetic field gradient (e.g., from one or more magnets 58, 108, 308 arranged as shown in FIGS. 1, 2, and 3), which can draw the clusters into the collection region (FIGS. 7 and 8). Detection can occur (e.g., using a light source 60, 110, 310 and a detector 62, 112, 312). As one example, light can be shined through the collection region (or just before the collection region) and any clusters can alter the intensity of the light so that a detector detects an intensity difference between light that passes through clusters and light that was shined through the sample before the magnetic field was applied to draw clusters into the collection region. In this example, the disease component can be determined to not be in the sample if the detected light has the same intensity as the previously shined light, while the disease component can be determined to be within the sample if an intensity change is detected (e.g., a decrease in intensity (light is blocked by cluster(s)) or an increase in intensity (due to the fluorescing of fluorescent materials attached to disease components). At step 810, the clusters, or disease components only, can be captured (e.g., for further analysis). The capture may occur with the one or more magnets moved or shielded so that the clusters are no longer subjected to the magnetic field gradient. In some instances, the functionalized magnetic particles (which may or may not be bound to the recognition component) can be recovered from the sample. As another example, a fluorescent dye can be attached to the disease component in the sample or the magnetic particle. The fluorescent dye can fluoresce when hit by the light and the presence of light emanating from the fluorescent dye is indicative of a presence of the disease component within the sample.

FIG. 10 illustrates another example method 900 for detecting and capturing certain disease components in a biological fluid. At step 902, a sample can be added to a sample holder (e.g., sample holder 52, 102, 302). The sample can include pre-added functionalized magnetic particles linked to one or more recognition components. Functionalized magnetic particles linked to one or more recognition components can also be added to the sample after the sample has been added to the sample holder, but before a magnetic field gradient is provided. Each of the one or more recognition components can be configured to bind to at least one disease component in the sample to form clusters. At step 904, a magnetic field gradient can be provided to the sample (e.g., by one or more magnets 58, 108, 308). The one or more magnets can be placed such that the magnetic field gradient can draw the clusters into a collection region of the sample holder. The magnetic field gradient can cause the clusters to be pulled into a collection region. The force of the magnetic field gradient can pull the clusters into the collection region without pulling in, or without substantially pulling in, non-clustered magnetic particles because the force on the clusters, which are significantly larger, is greater than the force on the single magnetic particles. The larger clusters, and any larger particles, are pulled into the collection region at a greater speed than smaller particles. At step 906, light can be shined through the collection region (e.g., collection region 56, 106, 306 of the sample holder 102, 302). The light can be shined through the collection region to detect clusters as the magnetic field gradient is being applied to draw any clusters into the collection region and/or after a time sufficient for all clusters (if any) to have already been drawn into the collection region. It should be understood that in some instances, the light can be shined through areas of the sample holder other than the collection region—e.g. the neck of the sample holder that the clusters must pass through to reach the collection region. A change in intensity of the light through the collection region is indicative of a presence of the disease component in the sample (e.g., the clusters block the light from reaching a detector or the clusters include a fluorescing agent that fluoresces in the light beam). The change in intensity of the light can be compared to light shown through the collection region before the magnetic field was applied. The functionalized magnetic particles (with or without the recognition component) can be collected after the test for the disease component, in some instances the magnetic field gradient can be removed before the collection. The disease components (or clusters containing the disease components) can also be collected after the detection for further analysis.

### EXAMPLE

COVID-19 is a disease caused by the virus SARS-CoV-2. Two types of test are available for COVID-19, a viral test (e.g., a nucleic acid amplification test or an antigen test) and an antibody test (e.g., a serology test). A viral test can determine whether a patient is currently infected. An antibody test can determine whether a patient has had a past infection. However, no device for detecting COVID can capture the viral particles or antibodies for further analysis like CAPTIV.

As an example, depending on the recognition component chosen, CAPTIV can be used to detect the SARS-CoV-2 virus or associated antibodies in a biological fluid and capture the SARS-CoV-2 virus or associated antibodies for further analysis. CAPTIV can serve a great need by determining the presence of an infection in a patient or an antibody response of the patient's immune system to help to control the spread of COVID-19. CAPTIV overcomes issues with the sensitivity of detection due to many obstacles, including the low amount of SARS-CoV-2 virus or associated antibodies in the amount of biofluid tested (which can lead to false negatives) or the long time after infection/exposure for SARS-CoV-2 virus or associated antibodies to reach a detectable level (when unknowing community spread can occur). Additionally, depending on how much biofluid needs to be processed, the CAPTIV procedures can be repeated multiple times to accommodate the large amount of biofluid.

Using CAPTIV, the SARS-CoV-2 virus or antibody can be detected and captured. For detection, coated magnetic particles can be added to a biofluid sample (including a sample taken from the patient, which may need to be diluted with a buffer like PBS or ultrafiltered water, or put into a fluid). The biofluid sample may also be tagged with a fluorescent substance for subsequent detection. It should be noted that the sample can be held in a sample holder, which has a collection region to collect the SARS-CoV-2 virus or antibody particles.

Before being added to the biofluid sample, the magnetic particles can be functionalized and then coated with a recognition component (e.g., viral receptors/ligands specific for SARS-CoV-2 or viral proteins specific for antibodies). The viral receptors/ligands specific for SARS-CoV-2 can include receptor Fc proteins, including ACE-2-Fc TMPRSS2-Fc, GRP-78-Fc DC-SIGN-Fc or DC-SIGNR-Fc. The viral proteins specific for antibodies can include native or recombinant proteinases, coat proteins, envelope proteins, or spike proteins. Both native and recombinant proteins, like M, E, S, N, HE, 3, 6, 7, 8, 9, 10, NSP and ORF proteins, viral associated proteins derived from infected cells, and RNA/DNA nucleic acid from the virus).

After placing these coated magnetic particles in a positive sample (and after waiting for a time period in which binding takes place and/or mixing the sample and magnetic particles, such as by rotation), the SARS-CoV-2 virus or associated antibodies can bind to the viral receptors/ligands or viral proteins (respectively) coating the magnetic particles. The viruses/antibodies are not themselves magnetic, but become magnetic when bound to the functionalized magnetic particles coated with viral proteins or viral receptors. A magnetic particle cluster can be formed (self-assembled) when the SARS-CoV-2 virus or associated antibodies bind to the ligands/viral proteins or viral receptors coating the magnetic particles. The force on a magnetic particle cluster because of a magnetic field gradient is far greater on multiple functionalized magnetic particles than that on an individual unbound functionalized magnetic particle coated with recognition component. This increased force can be used to move, concentrate, and capture the clusters. A light beam can traverse the collection region. As the clusters are drawn into the collection region, a greater proportion of the light beam is blocked, resulting in a decrease in intensity at the photodetector. As another option, the fluorescence can be triggered by the light beam, and the fluorescence can be detected at the photodetector.

After the detection (e.g., after a time period that allows for at least a majority of clusters to be pulled into the collection region), the magnetic field gradient can be removed (e.g., by moving or shielding the one or more magnets) so that the SARS-CoV-2 viruses or antibodies can be captured for further analysis. For example, when the collection region in a microfluidic channel, a PBS wash can be added to the sample holder and then withdrawn to remove nonmagnetic material and, following that, the concentrated SARS-CoV-2 viruses or antibodies can be isolated by removing the fluid (e.g., with a micropipette) and leaving only the clusters that can be captured for further analysis (either within the collection region or after transfer to another container). The analysis can be used as a patient-derived approach that allows analysis of the nuances or specificity of a potentially rapidly mutating virus or its associated antibodies in a particular patient.

While COVED-19 is one example disease and SARS-CoV-2 is one example virus, CAPTIV represents a platform that can be adapted to detect and capture any disease component where there is a recognition component is specific for the disease component.

References to “one aspect”, “an aspect”, “some aspects”, “one instance”, “an instance”, “some instances” “one example”, “an example”, “some examples” and so on, indicate that the embodiment(s) or example(s) so described may include a particular feature, structure, characteristic, property, element, or limitation, but that not every embodiment or example necessarily includes that particular feature, structure, characteristic, property, element, or limitation. Furthermore, repeated use of the phrase “in an aspect” does not necessarily refer to the same embodiment, though it may.

Where the disclosure or claims recite “a,” “an,” “a first,” or “another” element, the equivalent thereof, it should be interpreted to include one or more than one such element, neither requiring nor excluding two or more such elements. Furthermore, what have been described above are examples. It is, of course, not possible to describe every conceivable combination of components or methods, but one of ordinary skill in the art will recognize that many further combinations and permutations are possible. Accordingly, the invention is intended to embrace all such alterations, modifications, and variations that fall within the scope of this application, including the appended claims.

What is claimed is:

1. A system comprising:
a sample holder comprising:
a binding region configured to hold a sample and a plurality of functionalized magnetic particles, each of the plurality of functionalized magnetic particles being linked to a recognition component configured to bind to a disease component within the sample to form clusters; and
a collection region configured to collect and capture the disease component therein;
at least one magnet configured to provide a magnetic field gradient that draws the clusters from the binding region into the collection region;
a light source on one side of the collection region configured to shine a light beam through the collection region; and
a detector on an opposite side of the collection region from the light source configured to detect the light beam after the light beam has traversed the collection region to determine whether the disease component is present in the sample based on the detected light beam.

2. The system of claim 1, wherein the magnetic particles are indirectly linked to the recognition component through a linker molecule.

3. The system of claim 1, wherein the disease component comprises an antibody, a virus, a bacterium, a crystal, an exosome, or a cell from a cancerous tissue.

4. The system of claim 1, wherein the recognition component includes a viral protein, an envelope associated cellular protein, a proteinase, a coat protein, an envelope protein, a spike protein, an antibody, an antibody fragment, a peptide, or a nucleic acid.

5. The system of claim 1, wherein the recognition component includes an Fc chimera protein.

6. The system of claim 5, wherein the Fc chimera protein comprises ACE-2-Fc, TMPRSS2-Fc, GRP-78-Fc, DC-SIGN-Fc, or DC-SIGNR-Fc.

7. The system of claim 1, wherein the recognition component is a native and/or a recombinant protein, wherein the protein comprises one of M, E, S, N, HE, 3, 6, 7, 8, 9, 10, NSP and ORF proteins.

8. The system of claim 1, wherein the protein comprises a viral associated protein derived from infected cells.

9. The system of claim 1, wherein the recognition component comprises a nucleic acid including RNA and DNA.

10. The system of claim 1, wherein the disease component is a coronavirus that causes a disease, wherein the disease comprises COVID-19, SARS, or MERS.

11. The system of claim 1, wherein a size of the cluster allows capture in the collection region.

12. The system of claim 1, wherein a fluorescent dye is attached to the disease component and the light beam causes the fluorescent dye to fluoresce such that the detector detects light emanating from the fluorescent dye as indicative of the presence of the disease component within the sample.

13. The system of claim 1, wherein a change in an intensity of the detected light beam is indicative of the presence of the disease component within the sample.

14. A method comprising:
functionalizing magnetic particles to link to a certain recognition component, wherein the certain recognition component is configured to bind to a disease component;
linking a plurality of the functionalized magnetic particles to one or more of the certain recognition component;
adding the functionalized magnetic particles linked to the one or more of the certain recognition component to a sample holder that holds a sample, wherein each certain recognition component binds a disease component in the sample to form clusters;
drawing the clusters into a collection region of the sample holder with a magnetic field gradient; and
capturing the disease component in the collection region of the sample holder.

15. The method of claim 14 further comprising:
shining light through the collection region of the sample holder;
detecting the light that passes through the collection region of the sample holder; and
determining whether the disease component is present in the sample based on the detected light.

16. The method of claim 15, wherein the determining is based on a change in an intensity of the light through the collection region that is indicative of a presence of the disease component within the sample.

17. The method of claim 15, further comprising attaching a fluorescent dye to the disease component, wherein the fluorescent dye fluoresces when hit by the light and the presence of light emanating from the fluorescent dye is indicative of a presence of the disease component within the sample.

18. The method of claim 14, further comprising recovering the functionalized magnetic particles from the sample.

19. The method of claim 14, wherein the disease component comprises an antibody, a virus, a bacterium, a fungus, a crystal, an exosome, or a cell from a cancerous tissue.

20. A method comprising:
adding a sample to a sample holder, wherein the sample comprises functionalized magnetic particles linked to one or more recognition components, wherein each of the one or more recognition components are configured to bind to at least one disease component in the sample to form clusters;
providing a magnetic field gradient configured to draw the clusters into a collection region of the sample holder; and
shining light through the collection region of the sample holder,
wherein a change in intensity of the light through the collection region is indicative of a presence of the disease component in the sample.
